(19) United States
(12) Patent Application Publication
(10) Pub. No.: US 2003/0099600 A1
(43) Pub. Date: May 29, 2003

(54) Metalloproteinase inhibitors for the treatment of respiratory diseases

(76) Inventors: Andrew John McGlashan Richards, Essex (GB); Robin Mark Bannister, Essex (GB); Sharon Adele Chaplin, Essex (GB)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/227,101

(22) Filed: Aug. 23, 2002

Related U.S. Application Data
(63) Continuation-in-part of application No. PCT/GB01/00814, filed on Feb. 26, 2001.

(30) Foreign Application Priority Data
Feb. 25, 2000 (GB) 0004531.0

(51) Int. Cl. A61L 9/04; A61K 31/65

(52) U.S. Cl. 424/45; 514/152

(57) ABSTRACT

Doxycycline is useful for the treatment of a respiratory disease involving tissue destruction.

### REFERENCE TO RELATED APPLICATION

This Application is a continuation-in-part of PCT/GB01/00814, filed Feb. 26, 2001.

### FIELD OF THE INVENTION

This invention relates to the treatment of respiratory diseases.

### BACKGROUND OF THE INVENTION

Many respiratory diseases have acute components, which include reduction of gaseous exchange due to acute effects involving constriction of the airways. This may be due to infection, bronchoconstriction, excess mucous and other mechanisms. In addition, this is often accompanied by a more serious and irreversible destruction of lung tissue. These effects combined lead to a steady loss of lung function, resulting in lower quality of life and shortened life expectancy. Such diseases include chronic obstructive pulmonary disease (COPD), chronic bronchitis, emphysema, asthma, cystic fibrosis (CF) and lung cancer.

Matrix metalloproteinase enzymes are well known to have a central role in the tissue remodeling process. Inhibitors of these enzymes are under development for a number of therapeutic endpoints including inflammatory diseases (rheumatoid arthritis), oncology and periodontitis. Peptidic inhibitors of MMP enzymes have also been proposed for the treatment of lung diseases including COPD.

The tetracycline antibiotics are a well known class of compounds. They are normally administered as systemic antibiotics by the oral route. Typically, a tablet or capsule containing 50 mg or more of the drug is administered daily over a short period, in order to treat infection. In COPD, the underlying condition (which may have an infectious element) is typically treated using a bronchodilator.

Doxycycline and other tetracyclines are well known as moderate inhibitors of MMP enzymes. Doxycycline is registered, on the basis of this activity, for the treatment of periodontal disease.

U.S. Pat. No.5,773,430 discloses that certain hydrophobic tetracycline derivatives inhibit serine proteinases and also metalloproteinases. CF is included among the conditions that can be treated. Among the tetracyclines, “doxycline” (sic) is mentioned, but such compounds are considered as unsatisfactory by comparison with chemically modified tetracyclines and especially 4-de(dimethylamino)-tetracyclines.

U.S. Pat. No. 5,789,395 discloses that tetracycline compounds inhibit NO production. These compounds include doxycycline. The potential therapeutic uses may include respiratory diseases but not those include tissue destruction.

Oral doxycycline is extensively metabolised in the liver and, when given by a route of administration that avoids first-pass metabolism, lung concentrations of the drug can be up to 10 times the level in plasma; see Booker et al, Arzeneimittelforschung (1981)31(12):2116-7. In addition, when given by the oral route, doxycycline is known to be very highly protein-bound.

Systemic side-effects are described in BNF (September 2000) 264-5 and Physicians' Desk Reference ed.55 (2001) 1103 (Collagenex) and 2537 (Pfizer). Side-effects may be due to MMP inhibition and concomitant tissue effects.

### SUMMARY OF THE INVENTION

The present invention is based on studies that provides the first direct evidence concerning the utility of doxycycline as a modulator of MMP's and TIMP's, in the treatment of COPD. In particular, and surprisingly, it has been found that doxycycline modulates the levels of MMP enzymes when dosed to diseased lung tissue that has been resected from COPD patients with concurrent lung cancer. Dosing of doxycycline to this tissue reduces the levels of key MMP's implicated in tissue destruction in COPD. Doxycycline demonstrated a significant reduction in levels of MMP enzymes, when compared to control experiments. Decrease in MMP levels has been shown to lead to a direct correlation with the slowing of progression of tissue destruction in analogous connective tissue disorders, for example in arthritis and cancer metastases; see Shalinsky et al, Invest. New Drugs (1998-9) 16(4):303-13.

Surprisingly, in vivo, in addition to the effect upon MMP-9, it has now been demonstrated that doxycycline promotes significant increases in levels of TIMP-1, the natural inhibitor of MMP-9. These properties acting in concert allow doxycycline to exert potent MMP inhibitory activity, at doses effective for the treatment of tissue destruction-related diseases.

Particularly when doxycycline is administered according to the invention for the treatment of COPD, by the inhaled route, the benefit of the newly discovered MMP-modulating activity is maximised, and high concentration of drug in diseased tissue is encouraged. The exposure of drug in plasma, which may be lost through plasma binding, is minimised.

These factors work in concert to minimise overall exposure to drug and its unwanted side-effects. More particularly, according to the invention, and even when used by the oral route, owing to accumulation in the lung, the drug may be used at doses that are lower than for the treatment of other MMP-mediated diseases.

According to the present invention, doxycycline is used to treat a respiratory disease involving tissue destruction. More generally, based on the information provided herein, the active agent may be any that has an inhibitory activity of greater than 50% inhibition of MMP1 or MMP2 or MMP8 or MMP9 at less than 100 μM concentration in an enzyme assay and which also downregulates, in COPD lung tissue, MMP1 or MMP2 or MMP8 or MMP9 to less than 50% of untreated levels at 100 μM, and/or an inhibitory activity greater than 50% inhibition of MMP1 or MMP2 or MMP8 or MMP9 at a concentration of less than 100 μM in an enzyme assay which also upregulates TIMP-1 in COPD sputum to more than 200% of untreated levels following repeated dosing at 100 mg once daily.

The criticality of the roles of MMP and TIMP is illustrated by Vignola et al, Am. J. Respir. Core Med. (1998) 158(6):1945-50. See Segura-Valdez et al, Chest (March 2000) 117(3):684-94.

### DESCRIPTION OF PREFERRED EMBODIMENTS

A compound suitable for use in the invention may be readily determined by the skilled person, based on the standard assays and other information provided herein. For example, tetracyclines, including tetracycline antibiotics, are well known. Many such compounds have been disclosed and tested as antibiotics, and may be suitable for use in this invention. See also Mitscher, The Chemistry of the Tetracycline Antibiotics, Marcel Dekker, New York (1978), Chapter 6. Examples include doxycycline, tetracycline and minocycline.

A compound for use in the invention has one or both of the inhibitory profiles given above. In the first such profile, the given concentration is less than 100 μM, preferably less than 50 μM; for doxycycline, the value is about 20 μM. In the second profile, the upregulation is more than 200%, preferably more than 500%; for doxycycline, the value is about 1000%. Preferred compounds for use in the invention meet both these criteria, e.g. having at least one property that is at least as active as doxycycline.

An alternative expression of a compound suitable for use in the invention is that the compound is doxycycline, minocycline or a chemically modified tetracycline which exhibits metalloproteinase inhibitory activity and substantially no antimicrobial activity in a mammalian system. Such tetracyclines are described in U.S. Pat. No. 5,789,395 (the contents of this and other references given herein are incorporated by reference).

The preferred active agent for use in this invention is doxycycline, and this drug may be discussed below by way of illustration. Doxycycline may be used in the form of a salt, e.g. the hydrochloride.

Unlike many tetracyclines, doxycycline does not appear to accumulate in patients with impaired renal function, and aggravation of renal impairment may be less likely. As this invention may involve doxycycline being given on a chronic basis largely to older people have additional complications, including impaired renal function, this is a significant benefit over other drugs of its class.

Doxycycline is more lipid-soluble than tetracycline and accumulation in lipid-rich tissue when administered by the inhaled route may enhance efficacy. Doxycycline is known to accumulate in lung tissue, thereby setting up a concentration gradient in favour of the target tissue. Doxycycline is non-hygroscopic and is easy to control during formulation and dissolution when presented in a powder for reconstitution in a nebuliser product. For example, minocycline is hygroscopic.

Minocycline achieves higher drug concentrations in cerebrospinal fluid than doxycycline, thereby potentially leading to greater side-effects when administered in the chronic setting. Further, fewer bacterial strains are resistant to minocycline than doxycycline. Use of these more active antibacterial agents in a chronic setting in COPD will potentially lead to greater general resistance developing in the COPD lung. The long-term use of minocycline gives rise to significantly greater side-effect issues than doxycycline. These inclyde erythema nodosum, hepatitis, SLE and skin pigmentation. Hypersensitivity reactions may include athralgia, myalgia, pulmonary infiltration, and anaphylaxis. These side-effects appear less prevalent for doxycycline.

For use in this invention, the tetracycline antibiotic is formulated for inhalation or oral administration, and administered to a subject suffering from a respiratory disease involving tissue destruction The treatment may address the acute infectious element, but is also effective to treat the underlying tissue destruction that is present in some forms of lung disease, including COPD and CF. After a period of treatment, a reduction in infection is noted when the drug is administered at a dose appropriate as an anti-infective. At this dose and doses lower than necessary for use as an antibiotic, the rate of tissue destruction may also be reduced.

Modem methods of delivery of drugs by the inhaled route allow dosing to the lower lung. This may be achieved through control of particle properties (Including shape, size and electrostatic forces) using powder or liquid particle formulation. Suitable particle sizes are up to 1 μm, or up to 5 μm or above, depending on the intended target. Such control can be utilised to deliver doxycycline (by way of example) throughout the lung, and to the lower lung where, through its MMP inhibitory activity, it will slow and potentially reverse the rate of ongoing tissue destruction.

In particular, it has been found that it is possible to formulate tetracyclines in devices suitable for pulmonary delivery, and deliver them topically to the lung. This can be achieved using a range of pulmonary systems and formulation techniques known to those skilled in the art such as, but not limited to, for instance, nebulisers, multi-dose inhalers, dry powder inhalers and pressurised metered multi-dose inhalers. A tetracycline antibiotic such as doxycycline can be readily formulated for inhalation, e.g. with one or more conventional additives such as carriers, excipients, surface active agents etc.

The amount of the active agent to be administered will be determined by the usual factors such as the nature and severity of the disease, the condition of the patient and the potency of the agent itself. These factors can readily be determined by the skilled man. By way of example only, a suitable inhaled daily dose of doxycycline is 1 mg to 50 mg. The amount can be selected such that there is an antibiotic effect, if desired, or that there is no effective change in airway flora. More particularly for the latter purpose, the dosage per inhalation can be less than 20 mg, preferably less than 10 mg, e.g. less than 5 or even less than 2 mg.

Further, the active agent may also be administered by any oral route that provides appropriate drug concentration at the site of lung tissue destruction. Surprisingly, the MMP-lowering effect is seen at doses of below those customarily used to treat infection. Thus, doses below 50 mg (of doxycycline, or an equivalent amount of another suitable drug) can be used by the oral route to treat the tissue destruction seen in COPD. More generally, an oral dosage may be below 200 mg, often below 100 or 50 mg, and may even be below 25, 10, 5 or 1 mg. A suitable formulation for this purpose is a unit dosage such as a tablet or capsule.

The condition to be treated by means of the invention may be, for example, COPD, emphysema, asthma, CF or lung cancer. A related condition, i,e. chronic bronchitis, is essentially COPD when associated with tissue destruction. These are chronic conditions, and so treatment will generally be for longer than if infection only is treated. Treatment may be for at least 2 or 4 weeks, and generally for longer, e.g. months or even years.

It may be desirable to deliver tetracycline antibiotics to the lung in combination or concomitantly with other agents. These can be bronchodilators (e.g. beta-agonists such as salmeterol or terbutaline, or anticholinergics such as ipratropium), anti-inflammatories (e.g. steroids such as budesonide, beclomethasone or fluticasone, leukotriene antagonists and phosphodiesterase 4 inhibitors), anti-trypsin, or other anti-infective agents. In some cases, it may be desirable to formulate the drugs separately within an inhaler device, to achieve different release rates within the lung, dependent on the characteristics of the individual drugs at the site of action.

The studies on which the present invention is based will now be described. The data presented below demonstrate for the first time a multiple mechanism of action for doxycycline. The in vitro study shows the modest but useful inhibition of MMP-9 expression/secretion. The in vivo study demonstrates that doxycycline also increases the expression/secretion of the natural inhibitor of MMP-9 (TIMP-1). It is these two properties acting in concert that may allow doxycycline to exert surprisingly potent MMP inhibitory activity in the treatment of respiratory diseases.

Lung Tissue Study

This study was done to assess the effects of doxycycline on the release of matrix metalloproteinases by human lung tissue in vitro.

Lung tissue from a human who had a greater than 20 pack year history as a smoker, was chopped and incubated overnight in serum-free medium (RPMI 1640) containing penicillin, streptomycin and gentamycin (culture buffer). On the following day, 2-3 fragments (total weight 50 mg) were placed in 0.8 ml of culture buffer and 0.1 ml of doxycycline (to give a final concentration of 10⁻⁴-10⁻⁸M) or a buffer control was added.

After one hours incubation at 37° C., the fragments were treated with either 0.1 ml of either a buffer control (unstimulated fragments) or 1000 U/ml interleukin-1 (final concentration of 100 U/ml IL-1). The fragments were then incubated for 24 hours and the supernatant recovered; the tissue was weighed and stored at −70° C.

The MMPs and TIMPs released into the supematant were measured using commercial ELISAs (Amersham); values were expressed as ng of MMP/TIMP per mg of lung tissue.

Human Study

This was an open-label, ascending dose, cross-over study. Suitable subjects with chronic obstructive pulmonary disease (COPD) had their respiratory function assessed at baseline and provided sputum and blood for MMP and doxycycline levels. They then received 50 mg, 100 mg and 200 mg of doxycycline capsules for a period of 3 days in ascending order. At the end of each treatment period a further assessment of respiratory function was performed, and a sputum and blood sample was analysed for MMP, TIMP-1 and doxycycline levels. There was a 4-day wash out period between each treatment period of the study.

The MMPs and TIMPs released were measured using commercial ELISAs as detailed above for the lung tissue study.

MMP-1 Method Details

MMP-1 levels are analysed by Elisa plates (Code No. RPN2610) from Amersham Pharmacia Biotech UK Limited, Amersham Place, Little Chalfont, BUCKS HP7 9NA.

The Elisa is run as per manufacturer's instructions.

SPEC

This Elisa is specific for total MMP-1; recognising pro MMP-1, active MMP-1 and MMP-1/TIMP-1 complex.

Range: 6.25-100 ng/ml.

Sensitivity: 1.7 ng/ml.

Suitable for use with cell culture, serum and plasma samples.

Time: 5.5 hour protocol.

Store at −15 to −30° C.

MMP-2 Method Details

MMP-2 levels are analysed by Elisa plates (Code No. RPN2617) from Amersham Pharmecia Biotech UK Limited, Amersham Place, Little Chalfont, BUCKS HP7 9NA.

The Elisa is run as per manufacturer's instructions.

SPEC

This Elisa is specific for proMMP-2; recognising free proMMP-2 and MMP-2 complexed to TIMP-2, but not the active form of MMP-2. No cross-reactivity with MMP-1, 3, 7, 8, 9 and MT1-MMP.

Range: 1.5-24 ng/ml.

TABLE I
MMP-1 Lung Tissue Study

| % of control | | % of IL-1 | |
|---|---|---|---|
| +10 − 4 dox | 24.05 | +10 − 4 dox | 14.75 |
| +10 − 6 dox | 84.10 | +10 − 6 dox | 61.47 |
| +10 − 8 dox | 76.50 | +10 − 8 dox | 51.87 |

TABLE II
MMP-2 Lung Tissue Study

| % of control | | % of IL-1 | |
|---|---|---|---|
| +10 − 4 dox | 34.34 | +10 − 4 dox | 49.23 |
| +10 − 6 dox | 219.33 | +10 − 6 dox | 86.86 |
| +10 − 8 dox | 101.47 | +10 − 8 dox | 95.75 |

TABLE III
MMP-9 Lung Tissue Study

| % of control | | % of IL-1 | |
|---|---|---|---|
| +10 − 4 dox | 39.36 | +10 − 4 dox | 51.04 |
| +10 − 6 dox | 116.84 | +10 − 6 dox | 99.29 |
| +10 − 8 dox | 113.57 | +10 − 8 dox | 127.04 |

TABLE IV
TIMP-1 Lung Tissue Study

| % of control | | % of IL-1 | |
|---|---|---|---|
| +10 − 4 dox | 44.49 | +10 − 4 dox | 75.47 |
| +10 − 6 dox | 182.40 | +10 − 6 dox | 508.92 |
| +10 − 8 dox | 172.02 | +10 − 8 dox | 461.92 |

TABLE V
MMP-9 & TIMP-1 Human Study

% of control

| Day of Study | MMP-9 | TIMP-1 |
|---|---|---|
| 4 | 39.43 | 116.24 |
| 8 | 12.85 | 1101.03 |
| 11 | 29.17 | 1528.49 |
| 15 | 22.41 | 974.24 |
| 18 | 29.03 | 1173.36 |
| Mean | 26.58 | 978.67 |

The lung tissue study shows that lung tissue treated with 10⁻⁴ doxyeycline shows a dramatic decrease in MMP-1, 2 & 9.

The human patient study shows a 73% reduction in MMP-9 activity and a 10-fold increase in TIMP-1.

Modelling

Pharmacokinetic parameters for oral doxycycline are obtainable from the review of Saiuin and Houin, Clinical Pharmokinetics 15:355-366 (1988). Results given in Booker and Estler, Arzneim, Forsch. 31(II)12:2116-7 (1981), and Michel et al, Eur. J. Drug Metab. and Pharmacokin. 9(2)149-153 (1984), show that doxycycline is distributed from plasma into lung, and from there into the bronchial mucosa and secretions. As dosing continues, the exposure in sputum increases whereas that in plasma appears to have achieved equilibrium. As drug can only reach the sputum through the lung from the plasma and the plasma has reached equilibrium, the clearance from the lung must be slower than the apparent clearance from plasma.

Available data have been used to simulate a model for both serum and lung exposure.

The mean serum data from Saivin and Houin, supra, have been used to simulate a model for serum exposure based on a two-compartment elimination. As there are limited data for some parameters (AUC, Vd and Cl), more weight has been given to the Cmax, Tmax and half-life data. A similar approach has been taken to simulate lung exposure. The critiera used here were slightly late peak levels higher exposure in the lung compared with plasma at the later times a longer half-life in comparison with plasma and an overall exposure of approximately twice that in serum following a single dose.

Using these models, the pharmacokinetic parameters for both single and steady state dosing can be estimated. These data are shown in Table VI, assuming a dose interval of 24 hours.

TABLE VI
Multiple Dose (interval = 24 h) Parameters simulated for both serum
and lung

| Dose/Matrix | Cmax | Cmin | Tmax | AUC | No Doses |
|---|---|---|---|---|---|
| single serum | 3.6 | 0.5 | 2.6 | 28.7 | 1 |
| steady state serum | 4.1 | 0.7 | 2.3 | 36.6 | 5 |
| single lung | 2.3 | 1.4 | 4 | 42.4 | 1 |
| steady state lung | 4.6 | 2.8 | 3.2 | 88.8 | 8 |

Because of the longer half-life in lung, accumulation occurs and the exposure slowly increases with time relative to serum levels. According to this model, it would take 3 days longer to achieve steady state in the lung in comparison with plasma. This is in agreement with data on sputum levels discussed earlier. It is also in accord with the clinical practice of using a loading dose and various attempts to Increase the single dose to achieve maximum benefit in chronic bronchitis patients.

According to this model, the maximum concentration in the lung following a single dose would be 2.3 μg/g. At this point, the rate in is the same as the rate out, and therefore the maximum amount of drug in the lung occurs at this time. Assuming an average human lung weight of 1.2 kg, this is equivalent to 2.76 mg or approximately 1.5% of the 200 mg dose (assuming F_{abs}=0.9). On this basis, for an inhaled dose of approximately 3 mg applied directly to the lung, the lung exposure would be the same as an oral dose of 200 mg.

It is difficult to ensure complete dosing to the lung from an MDI device. The percentage of the dose entering the lung can be extremely variable and this must be taken into account when devising the dose. If 20% of the dose was directly available to the lung then the formulated dose should be of the order of 15 mg. Nevertheless, higher lung levels of doxycycline can be achieved through inhalation than by oral dosing.

The following Formulations illustrate how the invention may be practised.

Formulations

Formulations of doxycycline (sodium salt or free acid) are prepared in aqueous solution. The pH is adjusted to 3 to 5 to improve chemical stability, and isotonicity is achieved by addition of NaCl and/or dextrose.

Concentrations for clinical studies are at 1 mg/ml and 4 mg/ml, meaning single doses of doxycycline 5 mg and 20 mg, diluted in 5 ml reconstituion solution. The dosage form is dry powder to be reconstituted at the patient bedside with a tailor-made solution of NaCl, NaOH and optionally dextrose. The dosage is given by means of a nebuliser.

Observation shows that all solutions are stable after at least 24 h (chemically and physically), that there is no decrease of the drug concentration after 24 h at RT in an opened vial, even at a pH of 4.0, and that the solutions kept in a refrigerator for up to 1 month, also remained stable.

Satisfactory aerodynamic particle size distribution is achieved in vitro.

In a Pari LC Plus nebuliser, the full volume is 3 ml, the nebulisation time 8 min (until no solution comes out of the nebuliser), and the flow ACI 30 l/min. The fine particle dose is 5.47 mg doxycycline base. The fine particle fraction is 72.1% (with mouthpiece) and 75.5% (without mouthpiece). Thus, an effective dose can be achieved in this way.

1. A method for the treatment of a human subject who has a respiratory disease involving tissue destruction, which comprises administering to the subject an effective amount of doxycycline or a salt thereof.

2. The method according to claim 1, wherein the disease is a chronic condition.

3. The method according to claim 1, wherein the disease is selected from the group consisting of chronic obstructive pulmonary disease, emphysema, asthma, cystic fibrosis and lung cancer associated with chronic obstructive pulmonary disease.

4. The method according to claim 1, wherein the disease is essentially free of infection requiring an antibiotic.

5. The method according to claim 1, wherein the treatment does not affect airway flora.

6. The method according to claim 1, wherein the amount of doxycycline is sufficient to treat infection.

7. The method according to claim 6, wherein the amount of doxycycline is up to about 50 mg per dose.

8. The method according to claim 1, wherein the amount of doxycycline per dose is less than about 20 mg.

9. The method according to claim 1, wherein said amount is less than about 10 mg.

10. The method according to claim 1, wherein the administration is by inhalation.

11. The method according to claim 1, wherein the doxycycline is used in combination with or concomitantly with an additional agent selected from the group consisting of steroids, PDE 4 inhibitors, sympathomimetic agents, anti-cholinergics, bronchodilators, theophylline, elastase inhibitors, leukotriene antagonists, and anti-inflammatories.

12. The method according to claim 1, wherein the doxycycline is used in combination with another anti-infective agent.

13. A method for the treatment of tissue destruction in lung disease, which comprises the administration of an effective amount of doxycycline or a salt thereof.

14. A formulation of doxycycline or a salt thereof, suitable for administration via an inhalation device.

15. The formulation according to claim 14, in the form of a unit dose containing less than about 20 mg doxycycline.

16. The formulation according to claim 14, in the form of a unit dose containing less than about 10 mg doxycycline.
